(19) United States
(12) Patent Application Publication
(10) Pub. No.: US 2015/0265460 A1
(43) Pub. Date: Sep. 24, 2015

(54) INSTALLATION FOR GENERAL AIR CRYOTHERAPY

(71) Applicant: Khasanbi Khabievich Erganokov, Moscow (RU)

(72) Inventor: Khasanbi Khabievich Erganokov, Moscow (RU)

(21) Appl. No.: 14/406,458

(22) PCT Filed: Jun. 7, 2012
(86) PCT No.: PCT/RU2012/000443

§ 371 Date: Mar. 19, 2015

(51) Int. Cl. A61F 7/00

(52) U.S. Cl. CPC A61F 7/0053; A61F 2007/0096

(57) ABSTRACT

The proposed invention relates to the field of physical therapy and can be used for the treatment and prophylaxis of a number of diseases, and also for health-improving purposes, including improving the stamina, for instance, of athletes. The installation for conducting general air cryotherapy comprises a closed-type cryogenic chamber (1) with a door (2) with heat insulation (3) and a heat sensor (8), a refrigeration machine (7) with a condenser and a power and control unit (9), and a coolant supply line (6), wherein the refrigeration machine (7) has two cascades, and an evaporator (4) with a ventilator (5) is installed into the ceiling or wall of the cryogenic chamber (1) and is connected by means of the coolant supply line (6) to the double-cascade refrigeration machine (7), and wherein a heat control valve (12) is installed at the inlet to the evaporator (4), and the cryogenic chamber (1) is equipped with a valve for equalizing pressure with the outside environment (11), and wherein said valve (11) has an electric heater, the door (2) of the cryogenic chamber (1) is made entirely transparent and is equipped, as is the frame thereof, with an electric heater, and a control console with a sensor panel (13) is installed on the external surface of the cryogenic chamber (1). The proposed installation is more reliable than the prototype and the dimensions and cost of the installation are significantly decreased. The proposed installation can be used for the purposes of treatment and prophylaxis in the fields of rheumatoid arthritis, neurology, dermatology, traumatology and orthopedics, states of depression and stress, and in sports medicine.

### FIELD OF TECHNOLOGY

The proposed invention relates to the field of physical therapy and can be used for the treatment and prevention of a number of diseases, and also for health-improving purposes, including increasing the stamina of athletes, for example.

General air cryotherapy is a method of physical therapy based on the use of an air flow cooled to between −30° C. and −110° C. for cooling the human body, as a result of which the temperature of the body surface is lowered in the range of cryostability (5-10° C.) without pronounced shifts in the body's thermal regulation.

The action on the body of such low temperatures through the cold receptors of the skin within a short period of from 0.5 to 3 minutes makes it possible to obtain the following curative effects: elimination of acute and chronic pain; relief of inflammation, edema, spasm; regulation of muscle tone; improved respiratory functions; improved metabolism; strengthening of immunity; psychophysical training; improved well-being; restoration of regenerative and reproductive functions and youthfulness.

General air cryotherapy is a first-class means of training the physiological reserves of the human body and increasing resistance to stress and physical loads, the effect of the treatment being preserved after one round of cryo-procedures (10-15 sessions, one session every day or every other day) for at least 3-4 months.

### PRIOR ART

A system is known from European patent EP 0371072, having one or two pre-chambers, in which the temperature is maintained at intermediate levels, and through which the entry to and exit from the therapy chamber is effected.

A cryotherapy device is known from U.S. application No. 20100313579 (IPC: F25B19/00, F25D31/00), taken as the prior art for the proposed invention.

In this technical design, intended for general cryotherapy at a temperature of −85° C., the therapy chamber and the means of cooling with a three-stage compressor are installed on a portable, self-standing support structure. The means of cooling can include a condenser. Moreover, there are preparation chambers with intermediate temperature, maintained by means of temperature regulators. The system is powered from an electric generator. The control compartment includes means of regulating the temperature in the chambers and means for observation of the therapy procedure. There is provided a door for access from outside as well as a door with thermal insulation, connected to the therapy chamber and having a window allowing for observation of the situation inside the chamber.

The drawbacks of the device are unreliability due to the use of three compressors, a large footprint for stationary use on account of the pre-chambers being present, and high cost: the presence of a three-cascade machine, additional expenditure of coolant and oil for the three compressors; furthermore, the absence of elements inside the cryogenic chamber to equalize the pressure on the outside does not allow a reliable opening of the chamber doors.

### DISCLOSURE OF THE INVENTION

The problem to be solved by the invention is to significantly decrease the footprint, increase the reliability, and lower the installation and operating costs while ensuring a cryotherapy temperature regime of −85° C.

The problem is solved in that, in an installation for general air cryotherapy containing a closed-type cryogenic chamber with a heat sensor and a door with thermal insulation, a refrigeration machine with a condenser and a power supply and control unit, and a coolant supply line, the refrigeration machine has two cascades; there is introduced into the installation an evaporator with a ventilator or fan, installed in the ceiling or wall of the cryogenic chamber, and communicating with the refrigeration machine by way of the coolant supply line, wherein a heat-control valve is installed at the inlet of the evaporator; and the cryogenic chamber is provided with a valve for equalizing the pressure with the outside environment, said valve having an electric heater; the door of the cryogenic chamber is transparent, at least partly so, and is provided with electric heating, as is its frame; and a control console with a sensor panel is mounted on the outer surface of the cryogenic chamber.

### BRIEF DESCRIPTION OF THE FIGURES OF THE DRAWINGS

FIG. 1 and FIG. 2 show front and top views, respectively, of the proposed installation, where:

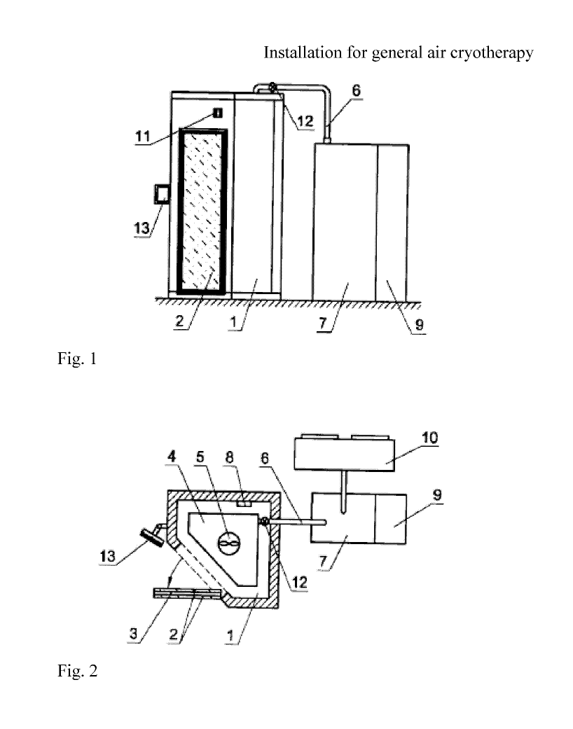

1—cryogenic chamber;

2—transparent door with electric heating of the surface and the frame;

3—heat insulation;

4—evaporator;

5—ventilator or fan;

6—coolant supply line;

7—double-cascade refrigeration machine;

8—heat sensor;

9—power supply and control unit;

10—condenser of refrigeration machine;

11—pressure-equalization valve with electric heating;

12—heat-control valve;

13—control console with sensor panel.

### PREFERRED EMBODIMENT OF THE INVENTION

The installation is composed of a closed-type cryogenic chamber 1, which is made of insulating materials and has a transparent door 2, at least partially, being made for example in the form of a glazing with heat insulation 3, with electric heating of the surface and frame of the door 2. The closed-type cryogenic chamber 1 can be made, for example, of rigid foam polyurethane panels, lined on both sides with sheets of galvanized metal with a powder coating based on polyvinyl chloride; the floor cover can be made of stainless steel sheeting with an anti-slip structure; inside the chamber on the ceiling there is mounted an evaporator 4 with a fan 5, while the ceiling below the evaporator is made of perforated stainless steel sheeting. A control console with a sensor panel 13 is mounted on an outside side panel, for example.

The installation contains a double-cascade refrigeration machine 7. The air in the chamber is cooled by means of the evaporator 4 and fan 5, arranged in the ceiling or wall of the cryogenic chamber 1. The evaporator 4 communicates with the double-cascade refrigeration machine 7 by means of the coolant supply line 6 via the heat-control valve 12, while the double-cascade refrigeration machine 7 is cooled down from overheating by a condenser 10 of the refrigeration machine, arranged for example behind the refrigeration machine 7. The entire installation is powered and controlled by a separate power supply and control unit 9.

The installation has a valve 11 for equalizing the pressure with the outside, with electric heating, which allows for an easy and reliable opening of the door of the cryogenic chamber 2 both from the outside and the inside, despite the temperature gradient of as much as 110 degrees between the outside and the inside surfaces of the cryogenic chamber 1.

A heat sensor 8 installed inside the cryogenic chamber 1 allows for monitoring and controlling the temperature conditions in the cryogenic chamber 1 by way of the power supply and control unit 9.

The presence of the transparent door 2 with electric hearing of the surface of the frame, made of glazing for example, prevents fogging of the glass surface of the door 2 for a reliable visual monitoring of what is occurring inside the cryogenic chamber 1 and creates extra psychological comfort for the patient undergoing the general air cryotherapy procedure.

The installation works as follows.

The installation is designed to work in an automatic mode. From the control console 13 the operator enters via the touch screen the operating parameters of the installation: the time to turn on and turn off the installation, for example, during the course of a week, the specified temperature range in the cryogenic chamber 1 during cryotherapy mode, the temperature in the cryogenic chamber 1 during standby mode, for example, at night, and the like.

Coolant from the double-cascade refrigeration machine 7 proceeds via the supply line 6 through the heat-control valve 12 to the evaporator 4, which is located in the cryogenic chamber 1; cooled air from the surface of the evaporator 4 is blown by the fan 5 into the volume of the cryogenic chamber 1, while the temperature inside it is monitored by means of the heat sensor 8.

The installation has the capability for changing the operating parameters, including in programmed manner.

### INDUSTRIAL APPLICABILITY

The proposed installation has greater reliability than the prior art, since it has no more than two cascades of the refrigeration machine for providing the specified cryotherapy temperature of −85° C., and it is also cheaper fewer cascades, and consequently fewer parts, less expenditure of oil for the compressors of the refrigeration machine, and, what is more, the footprint of the installation is significantly reduced thanks to the presence of only one cryogenic chamber.

The proposed installation can be used for curative and preventive purposes in the fields of rheumatology (rheumatic ailments of the soft tissues, rheumatic arthritis, Bekhterov's disease, arthroses, etc.), neurology (spinal pathology with pain syndrome), dermatology (systemic skin diseases), traumatology and orthopedics, depression and stress states, as well as sports medicine.

An experimental model of the installation has been constructed and has undergone successful testing.

1. An installation for general air cryotherapy containing a closed-type cryogenic chamber with a heat sensor and a door with thermal insulation, a refrigeration machine with a condenser and a power supply and control unit, a coolant supply line, is characterized in that the refrigeration machine has two cascades; there is introduced into the installation an evaporator with ventilator or fan, installed in the ceiling or wall of the cryogenic chamber, and communicating by way of the coolant supply line with the double-cascade refrigeration machine, wherein a heat-control valve is installed at the inlet of the evaporator; and the cryogenic chamber is provided with a valve for equalizing the pressure with the outside, said valve having an electric heater; the door of the cryogenic chamber is entirely transparent, and is provided with electric heating, including its frame; and a control console with a sensor panel is mounted on the outer surface of the cryogenic chamber.
